(19) United States
(12) Patent Application Publication
(10) Pub. No.: US 2006/0217385 A1
(43) Pub. Date: Sep. 28, 2006

(54) Nutritional preparations

(76) Inventors: John B. Edwards, Atlanta, GA (US); Lori T. Erlandson, Atlanta, GA (US); E. Nicholas Griffin, Cumming, GA (US); Alan T. Roberts, Woodstock, GA (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/372,239

(22) Filed: Mar. 9, 2006

Related U.S. Application Data
(60) Provisional application No. 60/660,419, filed on Mar. 10, 2005.

(51) Int. Cl. A61K 31/525; A61K 31/202

(52) U.S. Cl. 514/251; 514/560

(57) ABSTRACT

Compositions and methods for improving the nutritional and physiological status of a woman and her child during all stages of pregnancy are provided herein. This includes pre-conceptional women, pregnant women, and post-natal women (both lactating and non-lactating mothers). The compositions are particularly useful for the neurological, visual, and cognitive development of an embryo, fetus, or infant and the nutritional and physiological well-being of the mother, fetus, and infant. The compositions contain one or more folates, such as a reduced folate and/or folic acid, and one or more essential fatty acids (EFA), such as an omega-3 and/or omega-6 fatty acid. The addition of the essential fatty acid improves upon the folate containing nutritional preparations described in the prior art. The one or more folates and essential fatty acid may be administered together or in separate dosage units. The one or more folates may be selected from folic acid/folate, one or more reduced folates, or a combination of folic acid/folate and one or more reduced folates. The reduced folate is preferably 5-methyltetrahydrofolate, and most preferably 5-methyl-(6S)-tetrahydrofolic acid. The essential fatty acid is preferably an omega-3 fatty acid, and is preferably docosahexenoic acid (DHA) derived from a vegetarian or non-fish source. The compositions may optionally contain other vitamins, minerals, and ingredients, such as, emollient laxatives—all defined herein as “optional or other ingredients”.

### CROSS-REFERENCE TO RELATED APPLICATIONS

This application claims the benefit of U.S. Provisional Application No. 60/660,419, filed Mar. 10, 2005.

### FIELD OF THE INVENTION

The present invention relates generally to the field of nutrition, and more specifically to nutritional compositions and methods of neurological enhancement. The invention more specifically relates to nutritional supplements and methods for improving the nutritional and physiological status of women and their fetuses and children before, during, and after pregnancy.

### BACKGROUND OF THE INVENTION

Folate is an essential water-soluble B-vitamin that occurs naturally in food. Folic acid is the synthetic form of folate that is commonly found in supplements and added to fortified foods. In 1992, the U.S. Public Health Service recommended that all women of childbearing age consume 400 micrograms (0.4 μg) of folic acid daily. In addition, the U.S. Centers for Disease Control and Prevention (CDC) estimated that if all women of childbearing age took folate, the incidence of birth defects of the brain and spine could be reduced by as much as 70 percent. The recommended daily allowance of folate for males over 14 is 400 micrograms. In 1996, the U.S. Food and Drug Administration (FDA) published regulations requiring the addition of folic acid to enriched breads, cereals, flours, corn meals, pastas, rice, and other grain products. Since cereals and grains are widely consumed in the U.S. diet, these products have become an important contributor of folic acid to the American diet.

Folate helps produce and maintain new cells. This is especially important during periods of rapid cell division and growth such as infancy and pregnancy. Folate is needed to make DNA and RNA. It also helps prevent changes to DNA that may lead to cancer. Both adults and children need folate to make normal red blood cells and prevent anemia, including pregnancy-induced anemia. Folate is also essential for the metabolism of homocysteine and helps maintain normal levels of this amino acid. In a developing embryo or fetus, folate is required for DNA synthesis and repair, helping the baby's brain and spinal cord develop properly. However, folate must be available in the first 28 days of pregnancy to prevent neural tube defects (NTDs). Further evidence supports additional roles for folate in fetal development later in pregnancy, including the development of the heart, limbs and face. The risk of neural tube defects is significantly reduced when supplemental folic acid is consumed in addition to a healthy diet prior to and during the first month following conception.

Folate, commonly supplied as the synthetic form, folic acid, helps reduce the risk of a serious classification of birth defects known as neural tube defects. NTDs often include devastating abnormalities such as spina bifida and anencephaly. About one in every thousand pregnancies is afflicted with a NTD and an estimated 3,000 affected births per year in the United States were reported, prior to the introduction of the folate fortification program. Many additional affected pregnancies result in miscarriage or stillbirth. Spina bifida and anencephaly are severe central nervous system defects that result in serious disability and death. A worldwide estimate of affected pregnancies is approximately 300,000 to 400,000 neural tube defects annually. According to the CDC, approximately 50 to 70 percent of all NTD cases are preventable upon folate supplementation. This would effectively translate to the prevention of 150,000 to 200,000 NTDs worldwide each year.

The majority of infants afflicted with a neural tube defect will survive, but this is a life-altering congenital anomaly often leading to lower body paralysis and sensory loss, loss of bowel and bladder function, and hydrocephalus, which in turn may lead to multiple operations and hospitalizations. The average total lifetime cost for medical care for this disorder has been estimated to exceed a half a million dollars in many instances.

In contrast, anencephaly results when the upper portion of the neural tube fails to fuse. The majority of the brain and brain substance fails to form thus leading to a fatal condition in which a newborn is born with a severely underdeveloped brain and skull.

Folate is an essential nutrient for normal mammalian cell growth as a carrier of single carbon fragments. Reactions that utilize tetrahydrofolate as a donor of a single carbon unit include purine and pyrimidine synthesis as well as the provision of methyl groups for DNA, RNA and protein methylation. Folate deficiency has been shown to result in uracil misincorporation during DNA replication with subsequent increased double-strand breaks during uracil excision repair. Thus, an increased risk of leukemia-inducing translocations associated with low-folate status is conceivable.

Folate coenzymes are required for the metabolism of several important amino acids. The synthesis of methionine from homocysteine requires a folate coenzyme as well as a vitamin B₁₂ dependent enzyme. Thus, folate deficiency can result in decreased synthesis of methionine and a build up of homocysteine. Increased levels of homocysteine may be a risk factor for heart disease, as well as several other chronic diseases.

Folic acid undergoes a series of complicated vitamin and energy-dependent changes in the body, during transport between the intestine and liver, before it is converted to its active form, 5-methyl-(6S)-tetrahydrofolic acid (5-methyltetrahydrofolate). 5-methyltetrahydrofolate is the predominant form of folate in the human circulatory system and is the only type of folate that can cross the blood-brain barrier. Normal brain development and function depend on the active transport of 5-methyltetrahydrofolate across the blood-brain barrier.

The biologically active form of folate, 5-methyltetrahydrofolate (5-MTHF), may not be fully available to all women due to a common genetic mutation. A study published in 2000 by Botto and Yang of the Centers for Disease Control and Prevention, in the American Journal of Epidemiology (Botto, L. D., and Yang, Q. American Journal of Epidemiology, Vol 151, Issue 9: 862-877) demonstrated that one in eight women have a genetic trait that can prevent proper metabolism of folic acid. The trait is classified as homozygosity for the T allele of the C677T polymorphism of the gene encoding the folate dependent enzyme 5,10-methylenetetrahydrofolate reductase (MTHFR). It was reported by Botto and Yang that the homozygous genotype can be present in more than 40 percent of Hispanic women. The homozygous genotype was also observed in other ethnic subgroups. To date, the rate of NTDs remains higher in Hispanic women than in any other ethnic group.

As many as one in two women taking prenatal vitamins containing folic acid may not be capable of properly metabolizing folic acid into the form of folate needed to help prevent serious birth defects (Peng, F., Labelle, L. A., Rainey, B. J., Tsongalis, G. J. Int J Mol Med. 2001; 8: 509-511). In this study, the prevalence of a C677T or A1298C single nucleotide polymorphism (SNP) were investigated. Homozygosity for the C677T MTHFR SNP was detected in 16% and 10% of Caucasians and Hispanics, respectively. The frequency of the C677T heterozygous SNP for Caucasians and Hispanics was 56% and 52%, respectively.

A recent study by Kirke et al., (Kirke, P., Mills, J., Molloy, A., Brody, L., O'Leary, V., Daly, L., Murray, S., Conley, M., Mayne, P., Smith, O., Scott, J. BMJ, doi:10.1136/bmj.38036.646030EE (Published 21 May 2004)) confirmed that the 5-methyltetrahydrofolate polymorphism (C677T) of the gene encoding the folate dependent enzyme 5,10-methylenetetrahydrofolate reductase is a risk factor for neural tube defects. Moreover, Kirke et al., documented that up to 50% of folate-related NTDs can be explained by heterozygous or homozygous gene variants of this single mutation. The incidence and prevalence of MTHFR gene mutations of either homozygous or heterozygous genotype is much higher than previously supposed. These findings have important implications for pregnant and preconceptional women, public health, and women's health education.

The single nucleotide polymorphism C677T heterozygous genotype of the gene encoding the folate dependent enzyme MTHFR needs to be considered as a risk factor for other conditions where the homozygous genotype has been shown to be associated with an increased risk, such as cancer, including, but not limited to, colon cancer, breast cancer, head and neck carcinoma, thrombosis, schizophrenia, depression, dementia, inherited thrombophilia, hyperhomocysteinemia, preeclampsia, placental abruption, anemia, vascular disease and the like. Folate directed research over the last few years has clearly demonstrated common polymorphisms in folate dependent genes influence the risk of a number of diseases, not merely NTDs. For example, it is well documented that hyperhomocysteinemia is a risk factor for cardiovascular disease. Based on pooled data, research now suggests that about 59% of the European population and about 53% of the North American population have either CT or TT genotypes for the above SNP.

Unlike folic acid, the reduced folate, 5-methyltetrahydrofolate (5-MTHF) does not require enzymatic conversion to the biologically active compound. As described above, this enzymatic conversion process can be difficult for some individuals, especially those who carry a folate metabolic gene mutation. Therefore, compositions comprising a 5-MTHF eliminate, reduce or lessen the consequences of genetic deficiencies associated with folate metabolism. Such compositions incorporating 5-methyltetrahydrofolate and other folates are well-known and described in the art, for example, in U.S. Pat. Nos. 5,997,915; 6,011,040; 6,441,168; 5,350,851; and 6,921,754. These patents teach the use of nutritional preparations containing folate in pregnant females for the prevention of neural tube defects; females who have had a miscarriage; and females who have carried a fetus having a neural tube defect, a cleft lip defect, or a cleft palate defect.

While these preparations are beneficial, more improved compositions are needed to enhance the nutritional and physiological status of women during all stages of pregnancy and the neurological development of a fetus. There is also a need for compositions that improve the quality of breast milk and contribute to enhanced newborn visual, neurological and cognitive development. Additionally, there is a general overall need for a fundamentally new, safe, effective and comprehensive approach to address the treatment of folate deficiencies, the risk of NTDs in pregnant women, and aid those individuals affected with genetic mutations that compromise the folate metabolic pathway.

It is therefore an object of the present invention to provide improved compositions and methods for enhancing the neurological, visual, and cognitive development of an embryo, fetus, or infant and the nutritional and physiological well-being of the mother, unborn child, or infant before, during, and after pregnancy. It is a more specific object of the present invention to provide improved nutritional preparations containing folate, and particularly, reduced folate.

It is a further object of the invention to provide improved compositions for the treatment of men and women with a folate deficiency or a folate metabolic disorder and other conditions associated with folic acid deficiency.

### BRIEF SUMMARY OF THE INVENTION

Compositions and methods for improving the nutritional and physiological status of a woman and her child during all stages of pregnancy are provided herein. This includes pre-conceptional women, pregnant women, and post-natal women (both lactating and non-lactating mothers). The compositions are particularly useful for the neurological, visual, and cognitive development of an embryo, fetus, or infant and the nutritional and physiological well-being of the mother, fetus, and infant.

The compositions contain one or more folates, such as a reduced folate and/or folic acid, and one or more essential fatty acids (EFA), such as an omega-3 and/or omega-6 fatty acid. The addition of the essential fatty acid improves upon the folate containing nutritional preparations described in the prior art. The one or more folates and essential fatty acid may be administered together or in separate dosage units. The one or more folates may be selected from folic acid/folate, one or more reduced folates, or a combination of folic acid/folate and one or more reduced folates. The reduced folate is preferably 5-methyltetrahydrofolate, and most preferably 5-methyl-(6S)-tetrahydrofolic acid. The essential fatty acid is preferably an omega-3 fatty acid, and is preferably docosahexenoic acid (DHA) derived from a vegetarian or non-fish source. The compositions may optionally contain other vitamins, minerals, and ingredients, such as, emollient laxatives—all defined herein as “optional or other ingredients”.

The compositions are beneficial in the development of an embryo's central nervous system (brain and spinal cord). In particular, the compositions are used to aid in the development of the embryo's central nervous system between early embryological stage and late fetal development by administration of the claimed compositions to pregnant or preconceptional women.

The compositions are also directed to post-natal administration to lactating women, thereby supplementing the diet and providing adequate levels of essential vitamins, minerals and other nutrients to breast-fed newborns to aid continued growth and maturity of the brain, nervous system, retina and to assist in cognitive development. The compositions reduce or alleviate fetus- and infant-related folate deficiencies when administered to preconceptional women, pregnant, or post-natal women.

In addition, the compositions provide nutritional and physiological enhancement to women, especially women of childbearing age, preconceptional women or pregnant women by lowering the risk of developing a neural tube defect (NTD) and benefiting the neurological, visual, and cognitive development of an embryo or fetus. The compositions also help to prevent or reduce pregnancy-induced anemia.

Furthermore, the compositions can be administered to treat both women and men, having a folate deficiency or a folate metabolic disorder, or other conditions associated with folic acid deficiency. The compositions are especially helpful to individuals carrying a genetic mutation that inhibits, limits, reduces, or functionally restricts normal folic acid metabolism.

### DETAILED DESCRIPTION OF THE INVENTION

I. Compositions

The compositions described herein are compositions containing therapeutically effective amounts of one or more folates and one or more essential fatty acids (EFAs). As used herein, the phrase “one or more folates” is taken to mean that the compositions may contain 1. folic acid/folate; or 2. one or more reduced folates; or 3. folic acid/folate and one or more reduced folates. As used herein, the phrase “one or more essential fatty acids” is taken to mean that the compositions may contain 1. one or more omega-3 fatty acids including eicosapentaenoic acid (EPA), docosahexaenoic acid (DHA) and alpha-linolenic acid (ALA); or 2. one or more omega-6 fatty acids including linoleic acid (LA) and arachidonic acid (AA); or 3. a combination of one or more omega-3 fatty acids and one or more omega-6 fatty acids.

The compositions may optionally contain a therapeutically effective amount of one or more other ingredients such as vitamins, minerals and emollient laxatives. The phrase “an effective amount” or “a therapeutically effective amount” are used interchangeably and include an amount sufficient for 1) improving the nutritional and physiological status of a woman, fetus, or infant and the neurological, visual, and cognitive development of an embryo, fetus, or infant and/or 2) preventing, treating, or ameliorating a neural tube defect or one or more of the symptoms of folate deficiency or a folate metabolic disorder, or other conditions associated with folic acid deficiency and includes an amount which results in the effect that one or more of the symptoms of these disorders are ameliorated or otherwise beneficially altered.

As used herein “composition(s)” and “formulation(s)” are used interchangeably and include preparations such as multivitamins (with or without minerals and other nutrients); breakfast foods such as prepared cereals, toaster pastries and breakfast bars; infant formulas; dietary supplements and complete diet and weight-loss formulas and bars; animal feed (for example pet foods) and animal feed supplements (such as for poultry feed). As used herein, the term “nutritional preparation(s)” is encompassed by the terms “composition(s)” and “formulation(s)” and refers more specifically to multivitamin preparations (with or without minerals and other nutrients) and/or dietary supplements. Both the terms “compositions” and “nutritional preparations” can be taken to mean compositions and preparations where 1) the one or more folates and the one or more essential fatty acids are in the same dosage unit or 2) where the one or more folates and the one or more essential fatty acids are in separate dosage units. In one embodiment the one or more folates, one or more essential fatty acids, and optional other ingredients are contained in the same dosage unit. In a preferred embodiment, the one or more folates and one or more EFAs are in separate dosage units and given together as a single dose. In this embodiment, the other ingredients (vitamins, minerals, and emollient laxative) may be combined with either the one or more folates or the one or more EFAs. In a preferred embodiment, the optional other ingredients are combined with the one or more folates in a separate dosage unit from the one or more EFAs.

As described above, the compositions are beneficial for improving the nutritional and physiological status of a woman and her child during all stages of pregnancy. This includes pre-conceptional women, pregnant women, and post-natal women (both lactating and non-lactating mothers). As used herein, “nutritional status” refers to the presence or absence of any nutrient deficiency, the extent to which physiological nutrient demands are satisfied such that the deficiency is avoided.

The compositions are also particularly useful for the neurological, visual, and cognitive development of an embryo, fetus, or infant and the nutritional and physiological well-being of the mother, fetus, and infant. “Neurological development” refers to attainment of the highest degree of neurological development possible through natural processes without the use of any unnatural substances or procedures, such as drugs, surgery and the like.

The compositions are also useful for the treatment of men and women with a folate deficiency or a folate metabolic disorder and other conditions associated with folic acid deficiency such as, but not limited to, vascular disease, depression, hyperhomocysteinemia, thrombosis, pregnancy-induced thrombosis, pregnancy-induced anemia, neural tube defects, and homocysteine regulation. “Defective folate metabolic pathway” or “deficient folic acid metabolic pathway” or “folic acid metabolism disorder” refers to a less than normal, lack of, inhibited, or restricted production of folic acid pathway metabolites. The terms also refer to less than normal, deficient or defective levels of folic acid metabolites in a human or other animal.

A. Folates

As used herein, the term “folates” includes 1. folic acid, 2. the anionic form of folic acid, folate; and 3. natural and unnatural isomers of reduced folate or a pharmaceutically compatible salt or combination thereof. The compositions may contain one or more folates such as 1. folic acid/folate or 2. one or more reduced folates or 3. folic acid/folate and one or more reduced folates.

1. Reduced Folates

The term “reduced folate” is used herein to refer to both natural and unnatural isomers of reduced folate. Reduced folates and compositions containing these compounds are well-known and described in the art, for example, in U.S. Pat. Nos. 5,997,915; 6,011,040; 6,441,168; 5,350,851; and 6,921,754. Natural isomers of reduced folate suitable for use in the compositions include, for example, (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid. Other natural isomers of reduced folate include the polyglutamyl, such as the diglutamyl, triglutamyl, tetraglutamyl, pentaglutamyl, and hexaglutamyl, derivatives of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid.

Any or all of the natural isomers of reduced folate can be present in its chirally pure form, or, alternatively, the composition can optionally contain a molar amount of one or more unnatural isomers of reduced folate, such as (6R)-tetrahydrofolic acid, 5-methyl-(6R)-tetrahydrofolic acid, 5-formyl-(6S)-tetrahydrofolic acid, 10-formyl-(6S)-tetrahydrofolic acid, 5,10-methylene-(6S)-tetrahydrofolic acid, 5,10-methenyl-(6S)-tetrahydrofolic acid, 5-formimino-(6R)-tetrahydrofolic acid, and polyglutamyl derivatives thereof. The molar amount of the natural isomer of reduced folate can be equal to the molar amount of its corresponding unnatural isomer (as where the unnatural and natural isomer are present as a racemic mixture), or, preferably, the natural isomer of reduced folate can be present in a molar amount greater than the molar amount of the corresponding unnatural isomer. The total molar amount of the one or more natural isomers of reduced folate present in the composition can be between 5% and 200% of a human daily requirement for folate per a customarily consumed quantity of the composition. Natural isomers of reduced folates that are substantially chirally pure can be prepared by any suitable method, including, for example, by the method described in U.S. Pat. No. 5,350,851. Pharmaceutically compatible salts of the reduced folates may also be used in the compositions and should be both pharmacologically and pharmaceutically compatible salts such as, but not limited to, alkali or alkaline earth metal salts, preferably sodium, potassium, magnesium or calcium salts.

In a preferred embodiment, the reduced folate is 5-methyltetrahydrofolate. “5-methyltetrahydrofolate” is used herein to refer to the compound N-(5-methyl)-5,6,7,8-tetrahydropteroyl)-Lglutamic acid or a pharmaceutically acceptable salt thereof 1) as a racemate (5-methyl-(6R,S)-tetrahydrofolic acid), 2) in the form of the individual isomers, 5-methyl-(6R)-tetrahydrofolic acid and 5-methyl-(6S)-tetrahydrofolic acid, or 3) in a desired ratio of the individual isomers. 5-methyltetrahydrofolate can be used interchangeably with “5-methyl-tetrahydrofolic acid”, “5-methylTHF”, “L-methylfolate”, “L-methyltetrahydrofolate”. This compound is well-known and described in U.S. Pat. Nos. 5,997,915. Salt forms of this compound are also well-known and described in U.S. Pat. No. 6,441,168. In the most preferred embodiment, the 5-methyltetrahydrofolate is 5-methyl-(6S)-tetrahydrofolic acid.

Unlike folic acid, 5-methyltetrahydrofolate does not require enzymatic conversion to the biologically active compound. This enzymatic conversion process can be difficult for some individuals, especially those who carry a folate metabolic gene mutation. Clearly, the population at risk, and the population that can benefit from the presence of 5-methyltetrahydrofolate supplementation, is much larger than previously believed. In addition, those individuals affected by a genetic mutation in the folate metabolic pathway, especially those mutations that affect 5-methyltetrahydrofolate production or function, can be aided through the administration of a composition comprising 5-methyltetrahydrofolate. Therefore, compositions comprising 5-methyltetrahydrofolate and other reduced folates, eliminate, reduce or lessen the consequences of 5-methyltetrahydrofolate genetic deficiencies associated with folate metabolism.

5-methyltetrahydrofolate has been shown to be an ingredient of high bioavailability. In this application “bioavailable” and “bioavailability” are interchangeable and refer to “the degree to which, or rate at which, a drug or other substance is absorbed or becomes available at the site of physiological activity after administration”. Preliminary research suggests that 5-methyltetrahydrofolate is as equally bioavailable as folic acid. In particular circumstances, host-related factors, such as gastrointestinal illness and pH of the small intestine, can influence the bioavailability of folic acid, because it can be best converted into the active form prior to transport across the blood-brain barrier. Because of the concerns associated with folic acid bioavailability and the risk of NTDs, there is a need in the art for compositions and methods that improve embryonic or fetal neurological development. Furthermore, there is also a need for improved compositions to aid neurological development of a fetus during pregnancy or a breast-fed infant. For the reasons described above, compositions and nutritional preparations containing reduced folate in combination with an omega-3 fatty acid are more beneficial than those just containing folic acid and an omega-3 fatty acid, as described in U.S. Patent Publication No. 2003/0050341.

A study published in September 2003 by the March of Dimes organization found that less than one-third of American women of childbearing age took a daily multivitamin containing folic acid. As a consequence, by the time many women learn they are pregnant, the crucial period immediately following conception has already passed and any abnormal developments in the neural tube have already occurred.

As such, the compositions and methods provided herein are useful for reducing the risks associated with NTDs during pregnancy and aid the neurological development of a fetus prior to, during and after conception. In one embodiment, the compositions comprise a ready-to-use folate (i.e. reduced folate such as 5-methyltetrahydrofolate), which transports readily across the blood-brain barrier. Furthermore, compositions that contain the biologically active folate help alleviate the consequences of a genetically-induced folate metabolic disorder. In a specific embodiment, the compositions containing the reduced folate can alleviate folate metabolism deficiencies associated with MTHFR.

Therapeutically effective amounts of folate that may be used in the compositions and preparations described herein preferably ranges from 400 μg to 7 mg. In one embodiment, the amount of folate ranges from 500 μg to 4 mg. In a specific embodiment, the folate is 5-methyl-(6S)-tetrahydrofolic acid present in a range of from 600 μg to 1 mg.

2. Folic Acid

The compositions and preparations may comprise folic acid and/or the anionic form of folic acid, folate, in addition to, or in place of, the reduced folates described above. Compositions comprising, for example, both the reduced folate, 5-methyltetrahydrofolate, and folic acid have the increased benefit of providing a readily available form of biologically active 5-methyltetrahydrofolate while simultaneously providing a longer term source of folate, folic acid. As discussed above, folic acid must undergo enzymatic conversion to the biologically active form. Therefore, the combination of 5-methyltetrahydrofolate and folic acid provides a longer term source of folates than 5-methyltetrahydrofolate alone. Therapeutically effective amounts of folic acid that may be used in the compositions described herein preferably ranges from about 50 μg to about 6 mg. In another embodiment, the amount of folic acid present in the compositions described herein is about 200 μg and about 2 mg. In a specific embodiment, the amount of folic acid present in the compositions described herein is about 400 μg to about 1 mg. In another embodiment, wherein the compositions are administered to pregnant women as a prenatal supplement and contain folic acid, the amount of folic acid in the composition is at least 200 μg. In a preferred embodiment, wherein the compositions are administered to pregnant women as a prenatal supplement and contain folic acid, the amount of folic acid in the composition is about 400 μg to approximately 1 mg.

If both a reduced folate (e.g. 5-methyltetrahydrofolate) and folic acid are used in the compositions, the total amount of folate provided can be represented as the sum of the folate and folic acid. In one embodiment, the total amount of folate present in the compositions ranges from about 0% to about 40% folic acid, and about 60% to about 100% reduced folate. In a specific embodiment, the total amount of folate in the composition is about 400 μg to about 7 mg. In a preferred embodiment, the total amount of folate in the composition is about 1 mg to about 2 mg. In another embodiment, the total amount of folate is about 1 mg.

B. Essential Fatty Acids

One or more essential fatty acids (EFA), such as omega-3 or omega-6 fatty acids, are included in the folate-containing compositions described above. These compounds are well-known and described in the art, for example, in U.S. Patent Application Publication Nos. 2004/0082523 and 2002/0198177. The omega-3 and omega-6 fatty acids are polyunsaturated fatty acids classified as essential because humans cannot synthesize fatty acids and must obtain them through diet. The essential omega-3 fatty acids include eicosapentaenoic acid (EPA), docosahexaenoic acid (DHA) and alpha-linolenic acid (ALA). The human body possesses enzymes that convert ALA to EPA and DHA. Linoleic acid (LA) and arachidonic acid (AA) are examples of omega-6 fatty acids.

In a preferred embodiment, the EFA provided in the compositions is an omega-3 fatty acid or a mixture of omega-3 fatty acids, and preferably contains docosahexaenoic acid (DHA). DHA and vitamin/mineral compositions containing this essential fatty acid are described in detail in U.S. Patent Publication No. 2003/0050341. DHA is one of the main components of brain and heart tissue. It is required for the proper functioning of all neural systems, including the brain, the retina and the central nervous system. In clinical studies, DHA has shown the ability to increase the production of HDL, the so-called good cholesterol. DHA has also shown the ability to inhibit the production of pro-inflammatory prostaglandins. More recently, DHA has been shown to affect neurological function, learning capabilities and behavioral problems such as attention span and the ability to focus. Furthermore, DHA supplementation in infants is reported to facilitate cognitive growth and maturation. Indeed, recent studies report that essential fatty acids enhance fetal cognitive, visual and nervous system maturity. Therefore, it is desirable to administer compositions containing DHA to women of childbearing age to enhance the cognitive development of infants.

The brain and nervous tissue undergo a growth spurt from the last trimester of pregnancy to about the first 18 months of life. During this time, an adequate supply of fatty acids are needed to meet the needs of the developing fetus. DHA has been found to be essential for the healthy development of the cerebral cortex of the brain and retina in a baby as well as in an adult. It has been estimated that about half of the quantity of DHA in a fetus' body accumulates in the brain before birth and about half after birth, an indication of the importance of DHA to the fetus during pregnancy and then to the young infant during lactation. A fetus receives docosahexaenoic acid (DHA) from its mother while in the womb. After birth, breast-fed infants receive DHA and other fatty acids from the mother's breast milk and they are also able to make their own DHA. To that end, a mother taking supplements containing DHA during both pregnancy and after childbirth will elevate the DHA levels in her blood thereby supplying the baby. Thus, with regular DHA dosages, the baby will receive adequate levels of DHA for healthy neurological and vision development during prenatal and post-natal periods, and the depleted levels of DHA in the mother are restored.

In addition, the nutritional and health benefits of DHA for the mother are also desirable. For example, supplementation of DHA for a mother has been shown to help in the prevention of depression, including postpartum depression, after the baby is born. Further, the benefits and positive effects of DHA extend well past infancy and into childhood as well. For example, supplementation of DHA in the nutritional regimen of a child has been found to be desirable in the prevention of attention deficit/hyperactivity disorder in children.

In one embodiment, the compositions may contain DHA that is substantially free (<10%, and preferably <5%) of other Omega-3 fatty acids. In another embodiment, the DHA may be relatively free (<10%, and preferably <5%) of Omega-6 fatty acids, such as linoleic acid. In a preferred embodiment, the compositions contain linoleic acid in concentrations less than or equal to 5% by weight of DHA raw materials. In another embodiment, the DHA component of the compositions and preparations contains at least about 40% DHA relative to all other fatty acids. Furthermore, administering the compositions comprising DHA to a pregnant woman allows for the stable neurological development of an embryo or fetus.

DHA and EPA are mainly available as fish oil extracts. However, compositions containing fish-derived DHA, for example, may have a potent, offensive taste or odor. Additionally, substances derived from fish are believed to contain contaminants such as pollutants or ocean-borne contaminants, including dioxin and mercury, which can be detrimental to a developing embryo. Therefore, it is desirable to use DHA derived from a natural source, preferably a vegetarian or non-fish source. In a preferred embodiment, the compositions are formulated to comprise DHA derived from a vegetarian source such as, but not limited to, algae. In a specific embodiment, the compositions include DHA derived from the alga species Crypthecodinium cohnii. Methods for the production of DHA from algae are described in the following patents, U.S. Pat. Nos. 5,130,242, 5,340,742, 5,340,594, 6,451,567, 6,509,178, and 6,607,900.

The algae-derived DHA compositions satisfy the unmet needs of the prior art because the instant compositions are free from fish oil, fish matter, or other fish products. Both the U.S. Food and Drug Administration and the Environmental Protection Agency advise women who become pregnant, or are pregnant or nursing, to limit their intake of certain fish due to findings that certain fish contain significant levels of ocean-borne contaminants. The compositions of the instant invention provide additional benefit over the prior art because the compositions contain algae-derived DHA that is not derived from fish, thereby eliminating a fishy smell or taste that is often reported and associated with fish and fish oil products. This is a significant development for pregnant women who frequently suffer from nausea, that can be induced by potent or over-powering odors or tastes.

As stated above, the one or more EFAs can be contained within the same dosage unit as the one or more folates or may be in a separate dosage unit. In a preferred embodiment, the essential fatty acid is provided as a separate DHA capsule that is substantially free of other vitamins or minerals. The one or more EFAs may be presented in a hard capsule, such as, but not limited to, a hard gelatin capsule, or a soft gelatin (softgel) capsule. In one embodiment, the one or more EFAs are presented in an encapsulated semi-solid or liquid form. In a preferred embodiment, the EFA component is DHA presented in a semi-solid or liquid form packaged in a soft gelatin (softgel) capsule. In one embodiment, the soft gelatin capsule is prepared from vegetable or plant based materials. In a further embodiment, the soft gelatin capsules are made from cellulosic raw materials. In a preferred embodiment the soft gelatin capsules are preservative-free, easy to swallow, effectively mask taste and odor, and allow product visibility. Furthermore, in a specific embodiment, the DHA soft gelatin capsules prepared from plant or vegetable origins meet the strict dietary needs of individuals that choose vegetarian, as well as Kosher, lifestyles.

Therapeutically effective amounts of essential fatty acids that may be used in the compositions and preparations preferably range from about 100 mg to about 1 g. In one embodiment, the amount of the EFA present in the compositions and preparations ranges from about 200 mg to about 800 mg. In a specific embodiment, the essential fatty acid is DHA present in the compositions and preparations in a range of from about 250 mg to about 500 mg. In one embodiment, wherein the compositions comprise a DHA softgel capsule, the DHA softgel capsule may optionally contain DHA that is essentially free of other vitamins, minerals and Omega-3 fatty acids. In another embodiment, the DHA softgel capsule may optionally comprise a DHA softgel capsule that is essentially free of eicosapentaenoic acid and linolenic acid.

C. Other Vitamins, Minerals, and Ingredients

1. Iron

The compositions may optionally include an iron compound or derivatives thereof. In one embodiment, an effective amount of iron in the compositions ranges from about 10 mg to about 200 mg of iron compound or derivative. In one embodiment, the iron compound is elemental iron. In a preferred embodiment, the iron compound is carbonyl iron in a range of from about 80 mg to about 130 mg, and preferably 90 mg. In an alternative embodiment, the iron compound is an iron salt or combinations thereof, including, but not limited to, ferrous sulfate, ferrous fumarate, ferrous succinate, ferrous gluconate, ferrous lactate, ferrous glutamate or ferrous glycinate in a range of from 20 mg to 80 mg.

2. Copper

The compositions may optionally include a copper compound or derivatives thereof. Preferably, the amount of copper in the compositions ranges from about 0.1 mg to about 10 mg of copper compound or derivative. In one embodiment, the amount of copper in the compositions ranges from about 1 mg to about 5 mg. In a specific embodiment, the amount of copper in the compositions ranges from about 1.5 mg to about 2.5 mg. In one embodiment, the copper compound is cupric oxide.

3. Zinc

The compositions may optionally include a zinc compound or derivatives thereof. Preferably, the amount of zinc in the compositions ranges from about 5 mg to about 100 mg of zinc compound or derivative. In one embodiment, the amount of zinc in the compositions ranges from about 10 mg to about 30 mg. In a specific embodiment, the amount of zinc in the compositions ranges from about 12 mg to about 20 mg. In a preferred embodiment, the zinc compound is zinc oxide.

4. Magnesium

The compositions may optionally include a magnesium compound or derivatives thereof. Preferably, the amount of magnesium in the compositions ranges from about 5 mg to about 400 mg of magnesium compound or derivative. In one embodiment, the amount of magnesium in the compositions ranges from about 10 mg to about 200 mg. In a specific embodiment, the amount of magnesium in the compositions ranges from about 20 mg to about 100 mg. In a preferred embodiment, the magnesium compound is magnesium oxide. Biologically-acceptable magnesium compounds which may be incorporated into the present inventive subject matter include, but are not limited to, magnesium stearate, magnesium carbonate, magnesium oxide, magnesium hydroxide and magnesium sulfate.

5. Calcium

The compositions may optionally include a calcium compound or derivatives thereof. Preferably, the amount of calcium in the compositions ranges from about 20 mg to about 2500 mg of calcium compound or derivative. In one embodiment, the amount of calcium in the compositions ranges from about 150 mg to about 2000 mg. In a specific embodiment, the amount of calcium in the compositions ranges from about 175 mg to about 500 mg. Biologically-acceptable calcium compounds include, but are not limited to, any of the well known calcium supplements, such as calcium carbonate, calcium sulfate, calcium oxide, calcium hydroxide, calcium apatite, calcium citrate-malate, bone meal, oyster shell, calcium gluconate, calcium lactate, calcium phosphate, calcium levulinate, and the like.

6. Vitamin B₁

The formulations of the compositions described herein may optionally contain vitamin B₁ (thiamine mononitrate) or derivatives thereof. Derivatives of vitamin B₁ include compounds formed from vitamin B₁ that are structurally distinct from vitamin B₁, but that retain the active function of vitamin B₁. The vitamin B₁ may be present in a single form or in various different forms in combination within the present compositions. The amount of vitamin B₁ in the compositions preferably ranges from about 0.5 mg to about 50 mg. In one embodiment, the amount of vitamin B₁ in the compositions ranges from about 1 mg to about 4 mg. In a specific embodiment, the amount of vitamin B₁ in the compositions ranges from about 2 mg to about 3.5 mg.

7. Vitamin B₂

The formulations may optionally include vitamin B₂ (riboflavin) or derivatives thereof. Derivatives of vitamin B₂ include compounds formed from vitamin B₂ that are structurally distinct from vitamin B₂, but that retain the active function of vitamin B₂. The vitamin B₂ may be present in a single form or in various different forms in combination within the present compositions. The amount of vitamin B₂ in the compositions preferably ranges from about 0.5 μg to about 50 mg. In one embodiment, the amount of vitamin B₂ in the compositions ranges from about 1 mg to about 4.5 mg. In a specific embodiment, the amount of vitamin B₂ in the compositions ranges from about 3.0 mg to about 3.8 mg.

8. Vitamin B₆

The formulations may optionally contain vitamin B₆ (pyridoxine) or derivatives thereof. Derivatives of vitamin B₆ include compounds formed from vitamin B₆ that are structurally distinct from vitamin B₆, but that retain the active function of vitamin B₆. The vitamin B₆ may be present in a single form or in various different forms in combination within the present compositions. The amount of vitamin B₆ in the compositions preferably ranges from about 0.1 mg to about 200 mg. In one embodiment, the amount of vitamin B6 in the compositions ranges from about 2 mg to about 90 mg. In a specific embodiment, the amount of vitamin B₆ in the compositions ranges from about 10 mg to about 50 mg.

9. Vitamin B₁₂

The compositions may optionally include a vitamin B₁₂ or one of the three active forms: cyanocobalamin, hydroxocobalamin, or nitrocobalamin, or derivatives thereof. The derivatives of vitamin B₁₂ include compounds formed from vitamin B₁₂ that are structurally distinct from vitamin B₁₂, but that retain the active function of vitamin B₁₂. Non-limiting examples of such derivatives include methylcobalamin, deoxyadenosylobalamin, combinations thereof and the like. Preferably, the amount of vitamin B₁₂ in the instant compositions of the invention ranges from about 2 μg to about 250 μg. In one embodiment, the amount of vitamin B₁₂ in the compositions ranges from about 5 μg to about 30 μg. In a specific embodiment, the amount of vitamin B 12 in the compositions ranges from about 10 μg to about 20 μg.

10. Vitamin D₃

The formulations may optionally contain vitamin D₃ (cholecalciferol) or derivatives thereof. Derivatives of vitamin D₃ include compounds formed from vitamin D₃ that are structurally distinct from vitamin D₃, but that retain the active function of vitamin D₃. The vitamin D₃ may be present in a single form or in various different forms in combination within the present compositions. The amount of vitamin D₃ in the compositions preferably ranges from about 1 IU to about 2000 IU. In one embodiment, the amount of vitamin D₃ in the compositions ranges from about 200 IU to about 1500 IU. In a specific embodiment, the amount of vitamin D₃ in the compositions ranges from about 300 IU to about 1000 IU.

11. Vitamin E

The formulations may optionally include vitamin E (dl-alpha tocopheryl acetate) or derivatives thereof. Derivatives of vitamin E include compounds formed from vitamin E that are structurally distinct from vitamin E, but that retain the active function of vitamin E. The vitamin E may be present in a single form or in various different forms in combination within the present compositions. The amount of vitamin E in the compositions preferably ranges from about 1 IU to about 910 IU. In one embodiment, the amount of vitamin E in the compositions ranges from about 5 IU to about 500 IU. In a specific embodiment, the amount of vitamin E in the compositions ranges from about 8 IU to about 200 IU.

12. Vitamin C

The formulations described herein may optionally include vitamin C (ascorbic acid) or derivatives thereof. Derivatives of vitamin C include compounds formed from vitamin C that are structurally distinct from vitamin C, but that retain the active function of vitamin C. The vitamin C may be present in a single form or in various different forms in combination within the present compositions. The amount of vitamin C in the compositions preferably ranges from about 10 mg to about 2000 mg. In one embodiment, the amount of vitamin C in the compositions ranges from about 75 mg to about 1000 mg. In a specific embodiment, the amount of vitamin C in the compositions ranges from about 100 mg to about 500 mg.

13. Biotin

The formulations may optionally contain biotin or derivatives thereof. Derivatives of biotin include compounds formed from biotin that are structurally distinct from biotin, but that retain the active function of biotin. The biotin may be present in a single form or in various different forms in combination within the present compositions. The amount of biotin in the compositions preferably ranges from about 10 μg to about 50 μg. In one embodiment, the amount of biotin in the compositions ranges from about 20 μg to about 40 μg. In a specific embodiment, the amount of biotin in the compositions ranges from about 25 μg to about 35 μg.

14. Pantothenic Acid

The formulations may optionally include pantothenic acid (calcium pantothenate) or derivatives thereof. Derivatives of pantothenic acid include compounds formed from pantothenic acid that are structurally distinct from pantothenic acid, but that retain the active function of pantothenic acid. The pantothenic acid may be present in a single form or in various different forms in combination within the present compositions. The amount of pantothenic acid in the compositions preferably ranges from about 1 mg to about 10 mg. In one embodiment, the amount of pantothenic acid in the compositions ranges from about 3 mg to about 8 mg. In a specific embodiment, the amount of pantothenic acid in the compositions ranges from about 5 mg to about 7 mg.

15. Niacinamide

The formulations may optionally include niacinamide or derivatives thereof. Derivatives of niacinamide include compounds formed from niacinamide that are structurally distinct from niacinamide, but that retain the active function of niacinamide. The niacinamide may be present in a single form or in various different forms in combination within the present compositions. The amount of niacinamide in the compositions preferably ranges from about 1 mg to about 100 mg. In one embodiment, the amount of niacinamide in the compositions ranges from about 10 mg to about 30 mg. In a specific embodiment, the amount of niacinamide in the compositions ranges from about 15 mg to about 25 mg.

16. Vitamin A

The formulations may optionally include vitamin A from any commonly known source, for example, retinol or beta-carotene. Preferably, the source of vitamin A is beta-carotene. In one embodiment, vitamin A is provided in a total daily dose of between 0-10,000 IU, and preferably between 2,000 and 5,000 IU. However, in the most preferred embodiment, the compositions are formulated in the absence of vitamin A, especially when administered to pregnant women, since excess consumption of vitamin A is known to cause birth defects.

17. Emollient Laxatives

In one embodiment, the compositions optionally include an emollient laxative. The term “emollient laxative” is used herein to define a stool softener. In one embodiment, the emollient laxative is sodium docusate, glycerin, mineral oil or a poloxamer. In another embodiment, the emollient laxative is a pharmaceutically acceptable salt of docusate, such as, but not limited to, calcium. In another embodiment, the amount of emollient laxative provided in the instant compositions is between approximately 50 mg and approximately 1 g. In another embodiment, wherein the compositions are administered to pregnant women as a prenatal supplement the amount of emollient laxative in the composition is about 50 to about 200 mg. In a specific embodiment, wherein the compositions are administered to pregnant women as a prenatal supplement, the amount of emollient laxative in the composition is about 50 mg.

D. Salts and Derivatives

Although described above with reference specific to compounds, one can also utilize enantiomers, stereoisomers, derivatives and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418).

E. Formulations

In one embodiment, the one or more folates, one or more essential fatty acids (i.e. DHA), and optional ingredients such as vitamins, minerals, and emollient laxatives can be formulated in the same dosage unit. As used herein, “dosage unit” means any pharmaceutically acceptable form for administering a drug to a patient, including, but not limited to, capsules, tablets, buccal forms, troches, lozenges and oral liquids, suspensions or solutions. In a preferred embodiment, the one or more folates and optional ingredients are formulated into one dosage unit and the one or more EFAs are formulated in a separate dosage unit. In a preferred embodiment, the one or more folates and optional ingredients are formulated into a tablet, and the one or more essential fatty acids are formulated as a semi-solid or liquid in a separate softgel capsule. Softgels may be prepared, for example, without limitation, by dispersing the formulation in an appropriate vehicle to form a high viscosity mixture. This mixture is then encapsulated with a gelatin or vegetable based material using technology and machinery known to those in the softgel industry. The compositions comprising at least one tablet containing one or more folates and at least one softgel EFA capsule are presented together in one packing material. In a preferred embodiment, the at least one softgel EFA (e.g. DHA) capsule and the at least one tablet, are presented together within one blister-pack.

Film coated tablets, for example, without limitation, may be prepared by coating tablets using techniques such as, but not limited to, rotating pan coating methods or air suspension methods to deposit a contiguous film layer on a tablet. This procedure is often done to improve the aesthetic appearance of tablets, but may also be done to improve the ease of swallowing of tablets, or to mask an odor or taste. The compositions may conveniently be presented in unit dosage form and may be prepared by conventional pharmaceutical techniques. The compositions may be provided in a blister-pack or other such pharmaceutical package, without limitation. Further, the compositions may further include or be accompanied by indicia allowing a person to identify the compositions as products for women planning to conceive or who are pregnant. The indicia may further additionally include an indication of the above specified time periods for using said compositions.

Preferably, the compounds are orally administered. For oral administration, the compounds, particularly their acid addition salts, are formed into tablets, granules, powders or capsules containing suitable amounts of granules or powders by a conventional method together with usual drug additives. Oral formulations containing the active compounds may be in any conventionally used oral form, including tablets, capsules, buccal forms, troches, lozenges and oral liquids, suspensions or solutions. Oral formulations may utilize standard delay or time release formulations to alter the absorption of the active compound(s).

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The active compounds (or pharmaceutically acceptable salts thereof) may be administered in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, capsules, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose. Also suitable are hydrophobic polymers such as polyesters and polyimides. It is known to those skilled in the art that these polymers may be blended with polyanhydrides to achieve compositions with different drug release profiles and mechanical strengths. Preferably, the polymers are bioerodable, with preferred molecular weights ranging from 1000 to 15,000 kDa, and most preferably 2000 to 5000 Da.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Delayed release and extended release compositions can be prepared according to methods readily known in the art. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach

Examples of rate controlling polymers that may be used in the dosage form are hydroxypropylmethylcellulose (HPMC) with viscosities of either 5, 50, 100 or 4000 cps or blends of the different viscosities, ethylcellulose, methylmethacrylates, such as Eudragit RS100, Eudragit RL100, Eudragit NE 30D (supplied by Rohm America). Gastrosoluble polymers, such as Eudragit E100 or enteric polymers such as Eudragit L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

II. Methods of Use

A. Administration Protocol

The compositions of the present invention may involve the administration of the compositions at one or more times during a 24 hour period. For example, the compositions may be administered as a single dose of one or more tablets or capsules during a 24 hour period of time. In a preferred embodiment, the compositions are administered in a once daily dose.

The compositions are preferably administered prior to, during or after pregnancy. In one embodiment, the compositions and preparations are administered during a period of time commencing prior to conception and continuing through to completion of breast-feeding or continuing on as a nutritional supplement for the mother. The compositions and preparations may be given to both lactating and non-lactating mothers.

The compositions may be modified in dosage as required by one skilled in the art. In one embodiment, the dosage can be modified by one skilled in the art to treat or prevent a disease or disorder, or lessen the risks associated with a nutritional disorder. In a preferred embodiment, the dosage can be modified to treat a folate deficiency. In a specific embodiment, the dosage can be modified to provide preventative levels of folates, such as 5-methyltetrahydrofolate, to a woman who is planning to conceive or who is pregnant. In an alternative embodiment, the dosage can be modified to provide preventative levels of folates, such as 5-methyltetrahydrofolate, to a woman whose previous fetus developed a NTD in utero. In another embodiment, the dosage can be modified by one skilled in the art to provide adequate or normal levels of folates, such as 5-methyltetrahydrofolate, to a woman who is pregnant with multiple fetuses and thus requires increased levels of folate.

The methods are generally applicable to males and females unless expressively stated to the contrary. The methods are also applicable to healthy and ill individuals, and are particularly suitable for individuals with a folic acid deficiency or a genetic mutation within the folate metabolic pathway. Furthermore, the methods are applicable to preconceptional or pregnant women to reduce the risk of developing a NTD during pregnancy. In another embodiment, the methods are applicable to preconceptional, pregnant, or post-natal women to enhance the neurological and cognitive development of an embryo, fetus, or infant. In a specific embodiment, the methods of the invention are applicable as a prophylactic treatment of a disease or disorder associated with a folic acid deficiency in humans or other animals.

### EXAMPLES

### Example 1

A nutritional preparation including 5-methyltetrahydrofolate, DHA and optionally, a stool softener. A composition comprising the following constituents suitable for oral application is formed by: admixing 5-methyltetrahydrofolate, DHA and optionally, docusate sodium to form a mixture; and processing the mixture to form tablets or capsules; wherein the composition includes:

600 μg 5-methyltetrahydrofolate;

250 mg DHA; and

50 mg docusate sodium (optional)

### Example 2

A nutritional preparation including 5-methyltetrahydrofolate, DHA and optionally, folic acid a stool softener. A composition comprising the following constituents suitable for oral application is formed by: admixing 5-methyltetrahydrofolate, DHA and optionally, folic acid, and a stool softener to form a mixture; and processing the mixture to form tablets or capsules; wherein the composition includes.

600 μg 5-methyltetrahydrofolate;

400 μg folic acid (optional);

250 mg DHA; and

50 mg docusate sodium (optional)

### Example 3

A nutritional preparation including 5-methyltetrahydrofolate, DHA and optionally, folic acid, calcium, and a stool softener. A composition comprising the following constituents suitable for oral application is formed by: admixing 5-methyltetrahydrofolate, DHA, and optionally folic acid, calcium, and a stool softener to form a mixture; and processing the mixture to form tablets or capsules; wherein the composition includes:

600 μg 5-methyltetrahydrofolate;

400 μg folic acid (optional); 200 mg calcium (optional);

250 mg DHA; and

50 mg docusate sodium (optional).

### Example 4

A nutritional preparation including 5-methyltetrahydrofolate, DHA, and optionally folic acid, iron, and a stool softener. A composition comprising the following constituents suitable for oral administration is formed by: admixing 5-methyltetrahydrofolate, DHA, and optionally folic acid, iron, and a stool softener to form a mixture; and processing the mixture to form tablets or capsules; wherein the compositions includes:

600 μg 5-methyltetrahydrofolate;

400 μg folic acid (optional);

90 mg iron (optional);

250 mg DHA; and

50 mg docusate sodium.

### Example 5

A nutritional preparation including 5-methyltetrahydrofolate, DHA, and optionally folic acid, vitamin D₃, and a stool softener. A composition comprising the following constituents suitable for oral application is formed by: admixing 5-methyltetrahydrofolate, DHA, and optionally folic acid, vitamin D₃, and a stool softener to form a mixture; and processing the mixture to form tablets or capsules; wherein the composition includes:

600 μg 5-methyltetrahydrofolate;

400 μg folic acid (optional);

400 IU Vitamin D₃ (optional):

250 mg DHA; and

50 mg docusate sodium (optional).

### Example 6

A nutritional preparation including 5-methyltetrahydrofolate, DHA and optionally a stool softener. A composition comprising the following constituents suitable for oral application is formed by: admixing 5-methyltetrahydrofolate, DHA and optionally, docusate sodium to form a mixture; and processing the mixture to form tablets or capsules; wherein the composition includes:

1000 μg 5-methyltetrahydrofolate;

250 mg DHA; and

50 mg docusate sodium (optional).

In all of Examples 1-6 above, DHA may be provided separately from the other ingredients for example, in a soft gel capsule, as described in detail in the preceding sections.

### Example 7

The following nutritional preparation may be used for administration to preconceptional and pregnant women to reduce the risk of neural tube defects during pregnancy and to enhance the neurological and cognitive development of an embryo or fetus. It may also be used to improve the nutritional status of the woman throughout pregnancy and in the postnatal period for both lactating and non-lactating mothers. In addition, the preparation may be used for administration to breast feeding mothers to provide newborns with essential vitamins and nutrients to aid in continued growth and maturity of the brain, nervous system, and retina and to assist in cognitive development. Furthermore, it may be used to prepare preconceptional supplement products for administration to preconceptional women to improve the nutritional status of the woman prior to conception.

A preparation containing:

| | | |
|---|---|---|
| Elemental Iron | 10-200 | mg; |
| Biotin | 10-50 | mcg; |
| Pantothenic acid | 1-10 | mg; |
| Calcium | 20-2500 | mg; |
| Copper | 0.1-10 | mg; |
| Zinc | 5-100 | mg; |
| Folate | 400-7,000 | mcg; |
| Vitamin D₃ (cholecalciferol) | 1-2,000 | IU; |
| Vitamin E (dl-alpha tocopheryl) | 1-910 | IU; |
| Vitamin C (ascorbic acid) | 50-2000 | mg; |
| Vitamin B₁ (thiamine) | 0.5-50 | mg; |
| Vitamin B₂ (riboflavin) | 0.5-50 | mg; |
| Vitamin B₆ (pyridoxine) | 0.1-200 | mg; |
| Vitamin B₁₂ (cyanocobalamin) | 2-250 | mcg; |
| Niacinamide | 1-100 | mg; |
| Magnesium | 5-400 | mg; |
| Docusate Sodium, USP | 50-200 | mg. |
| DHA | 100-1,000 | mg. |
| Betacarotene (optional) | 0-10,000 | IU |

The DHA may be provided in the same dosage unit or a separate dosage unit as the other vitamins, minerals, and ingredients. In one preferred embodiment, the nutritional preparation does not contain betacarotene.

### Example 8

A similar nutritional preparation as that described in Example 7 with the preferred amounts of the vitamins, minerals, and ingredients is as follows. Metafolin® (5-methyl-(6S)-tetrahydrofolic acid, calcium salt; 1-methylfolate) is commercially available from Merck Eprova AG (Schaffausen, Switzerland).

A tablet containing:

| | | |
|---|---|---|
| Elemental Iron (carbonyl iron) | 90 | mg; |
| Biotin | 30 | mcg; |
| Pantothenic acid (calcium pantothenate, USP) | 6 | mg; |
| Calcium (calcium carbonate, USP) | 200 | mg; |
| Copper (cupric oxide) | 2 | mg; |
| Zinc (zinc oxide, USP) | 15 | mg; |
| Metafolin ® | 600 | mcg; |
| Folic acid, USP | 400 | mcg; |
| Vitamin D₃ (cholecalciferol) | 400 | IU; |
| Vitamin E (dl-alpha tocopheryl acetate) | 10 | IU; |
| Vitamin C (ascorbic acid, USP) | 120 | mg; |
| Vitamin B₁ (thiamine mononitrate) | 3 | mg; |
| Vitamin B₂ (riboflavin, USP) | 3.4 | mg; |
| Vitamin B₆ (pyridoxine HCl) | 20 | mg; |
| Vitamin B₁₂ (cyanocobalamin) | 12 | mcg; |
| Niacinamide, USP | 20 | mg; |
| Magnesium (magnesium oxide, USP) | 30 | mg; |
| Docusate Sodium, USP | 50 | mg. |

A soft gel capsule containing:

| | |
|---|---|
| DHA | 250 mg. |

Compositions incorporating the above formulation are prepared using conventional methods and materials known in the pharmaceutical art. The resulting folate supplements are recovered and stored for future use.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A method of improving the nutritional status of a woman throughout pregnancy and in the postnatal period comprising administering a therapeutically effective amount of one or more folates and one or more essential fatty acids to the woman prior to, during, or following pregnancy.

2. The method of claim 1, wherein the one or more folates and the one or more essential fatty acids are administered in the same dosage unit.

3. The method of claim 1, wherein, the one or more folates and one or more essential fatty acid are administered in separate dosage units.

4. The method of claim 3, wherein the separate dosage units are administered at the same time.

5. The method of claim 1, wherein the one or more folate is selected from the group consisting of folic acid, reduced folates, and a mixture thereof.

6. The method of claim 1, wherein the one or more essential fatty acids is selected from the group consisting of eicosapentaenoic acid (EPA), docosahexaenoic acid (DHA), alpha-linolenic acid (ALA), and mixtures thereof.

7. The method of claim 1, wherein the one or more esstential fatty acids is docosahexaenoic acid (DHA) that is substantially free of other omega-3 fatty acids.

8. A method for promoting the development of the central nervous system of an embryo or fetus between early embryological stage and late fetal development comprising administering a therapeutically effective amount of one or more folates and one or more essential fatty acids to a preconceptional or pregnant woman carrying the embryo or fetus.

9. The method of claim 8, wherein the one or more folates and one or more essential fatty acids are administered in the same dosage unit.

10. The method of claim 8, wherein, the one or more folates and one or more essential fatty acids are administered in separate dosage units.

11. The method of claim 10, wherein the separate dosage units are administered at the same time.

12. The method of claim 8, wherein the one or more folate is selected from the group consisting of folic acid, reduced folates, and a mixture thereof.

13. The method of claim 8, wherein the one or more essential fatty acids is selected from the group consisting of eicosapentaenoic acid (EPA), docosahexaenoic acid (DHA), alpha-linolenic acid (ALA), and mixtures thereof.

14. The method of claim 8, wherein the one or more esstential fatty acids is docosahexaenoic acid (DHA) that is substantially free of other omega-3 fatty acids.

15. A method for promoting the development of the central nervous system of an infant nursing from a lactating female comprising administering to the lactating female a therapeutically effective amount of one or more folates and one or more essential fatty acids.

16. The method of claim 15, wherein the one or more folate is selected from the group consisting of folic acid, reduced folates, and a mixture thereof.

17. The method of claim 15, wherein the one or more essential fatty acids is selected from the group consisting of eicosapentaenoic acid (EPA), docosahexaenoic acid (DHA), alpha-linolenic acid (ALA), and mixtures thereof.

18. The method of claim 15, wherein the one or more esstential fatty acids is docosahexaenoic acid (DHA) that is substantially free of other omega-3 fatty acids.

19. A method for reducing the risk of a fetus developing a neural tube defect in utero comprising administrating to a woman prior to, or during pregnancy, a therapeutically effective amount of one or more folates and one or more essential fatty acids.

20. The method of claim 19, wherein the one or more folate is selected from the group consisting of folic acid, reduced folates, and a mixture thereof.

21. The method of claim 19, wherein the one or more essential fatty acids is selected from the group consisting of eicosapentaenoic acid (EPA), docosahexaenoic acid (DHA), alpha-linolenic acid (ALA), and mixtures thereof.

22. The method of claim 19, wherein wherein the one or more esstential fatty acids is docosahexaenoic acid (DHA) that is substantially free of other omega-3 fatty acids.
